(19) United States
(12) Patent Application Publication
(10) Pub. No.: US 2026/0151059 A1
(43) Pub. Date: Jun. 4, 2026

(54) RESET PROCEDURE FOR STABLE REFERENCE

(71) Applicant: Medtronic, Inc., Minneapolis, MN (US)

(72) Inventors: Anna Marie Tycon, Scottsdale, AZ (US); Mohsen Askarinya, Chandler, AZ (US); Omid Mahdavi, Fountain Hills, AZ (US); Luling Wang, Mason, OH (US)

(21) Appl. No.: 19/122,418

(22) PCT Filed: Oct. 17, 2023
(86) PCT No.: PCT/US2023/035313

§ 371 Date: Apr. 18, 2025

Related U.S. Application Data
(60) Provisional application No. 63/417,396, filed on Oct. 19, 2022.

(51) Int. Cl. A61B 5/1473

(52) U.S. Cl. CPC A61B 5/1473; A61B 2562/04

(57) ABSTRACT

Analyte sensor apparatus, systems, and methods to reduce sensor drift. An analyte sensor apparatus (102) for detecting an analyte such as glucose in a target environment (10) such as a body tissue may include a plurality of electrodes and a controller. The plurality of electrodes may provide a plurality of electrode signals based on the target environment while disposed in the target environment. The controller may be operatively coupled to the plurality of electrodes and configured to receive the plurality of electrode signals. The controller may further be configured to adjust a voltage of at least one electrode of the plurality of electrodes based on a voltage profile to regenerate one or more electrodes of the plurality of electrodes while the at least one electrode is disposed in the target environment. This may allow to correct an electrode drift without removing the electrode from its target environment.

### RELATED APPLICATIONS

This application claims the benefit of U.S. Provisional Application Ser. No. 63/417,396 filed Oct. 19, 2022, which is incorporated by reference herein in its entirety.

### FIELD

The present disclosure relates to, among other things, analyte sensors.

### TECHNICAL BACKGROUND

Analyte sensors may be used in a wide range of applications such as, for example, point-of-care monitoring, environmental monitoring, food control, drug discovery, forensics, biomedical research, etc. Analyte sensors may be used to detect a substance of interest (e.g., an analyte) in a target environment. Analyte sensors generally include biotransducers (e.g., electrodes) to provide signals that can be used to determine the presence of an analyte in the target environment. Such biotransducers may be subject to noise in the target environment, sensor drift, reference variation, or other effects that can impact sensor response and accuracy.

To ensure accurate sensing of an analyte in a target environment, analyte sensors may be periodically calibrated or replaced. However, in some applications, calibration or replacement of the analyte sensors may be burdensome and expensive.

### BRIEF SUMMARY

As described herein, accurate and reliable analyte sensing can be achieved by adjusting a voltage of one or more electrodes of a sensor apparatus while such electrodes remain in a target environment. Generally, analyte sensors include a plurality of electrodes to provide a plurality of signals that may be used together to determine the presence of an analyte in a target area. However, conditions in the target environment, applied currents, and or applied voltages may cause the electrode signals to drift or increase the measurement error of the electrodes. To correct for such effects, a current or a voltage of one or more electrodes may be applied or adjusted in a manner that resets surface conditions of at least one of the electrodes to correct or reduce sensor drift and or measurement error of the at least one electrode.

Described herein, among other things, is an analyte sensor apparatus for detecting an analyte in a target environment comprising a plurality of electrodes and a controller. The plurality of electrodes may provide a plurality of electrode signals based on the target environment while disposed in the target environment. The controller may be operatively coupled to the plurality of electrodes and configured to receive the plurality of electrode signals. The controller may further be configured to adjust a voltage of at least one electrode of the plurality of electrodes based on a voltage profile to regenerate one or more electrodes of the plurality of electrodes while the at least one electrode is disposed in the target environment.

In general, in one aspect, the present disclosure describes an analyte sensing system comprising an analyte sensor apparatus for detecting an analyte in a target environment and a computing apparatus. The analyte sensor apparatus may comprise a plurality of electrodes to provide a plurality of electrode signals based on the target environment while disposed in the target environment. The computing apparatus may comprise one or more processors and may be operatively coupled to the analyte sensor apparatus. The computing apparatus may be configured to receive the plurality of electrode signals and adjust a voltage of at least one electrode of the plurality of electrodes based on a voltage profile to regenerate one or more electrodes of the plurality of electrodes while the at least one electrode is disposed in the target environment.

In general, in another aspect, the present disclosure describes a method to reduce sensor drift comprising receiving a plurality of electrode signals from a plurality of electrodes; and adjusting a voltage of at least one electrode of the plurality of electrodes based on a voltage profile to regenerate one or more electrodes of the plurality of electrodes while the at least one electrode is disposed in the target environment.

Advantages and additional features of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### BRIEF DESCRIPTION OF THE DRAWINGS

The following detailed description of specific embodiments of the present disclosure can be best understood when read in conjunction with the following drawings, in which:

FIG. 1 is a schematic diagram of an embodiment of an analyte sensing system;

FIG. 2 is a schematic block diagram of the analyte sensing system of FIG. 1;

FIG. 3 is a schematic block diagram another embodiment of an analyte sensing system; and

FIG. 4 is a flow diagram of an embodiment of a process to reduce sensor drift in a target environment.

The schematic drawings are not necessarily to scale.

### DETAILED DESCRIPTION

Reference will now be made in greater detail to various embodiments of the subject matter of the present disclosure, some embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

Sensor apparatus and systems may include a plurality of electrodes to provide signals used to determine the presence of an analyte in a target environment. Generally, the signals provided by such electrodes are directly related to the surface properties and charge state of such electrodes. Changes in the surface properties and signals may be used to detect and measure the analyte (e.g., glucose concentration in tissue) in the target environment. However, other factors in the in the target environment, applied currents, and/or applied voltages can also cause changes in the electrode surface properties and charge states over time that may cause signal drift that is unrelated to the analyte the electrodes are intended to measure. Typically, frequent calibrations and predictive algorithms (not including, e.g., artificial intelligence platforms and machine learning algorithms) are used to compensate for sensor drift. However, calibrations may require sensor apparatus to be removed from the target environment and may be cumbersome and inconvenient. Additionally, predictive algorithms may not account for unexpected variations of electrode surface properties during actual use.

Accurate and reliable analyte sensing can be achieved using analyte sensors and/or systems that can adjust a voltage of at least one electrode to regenerate one or more electrodes. The application or adjustment of a voltage applied to at least one electrode may allow the surface properties of electrodes to be reset or regenerated to correct sensor drift or measurement error of the electrodes. The voltage may be applied or adjusted based on a voltage profile. The voltage profile may include an application of one or more positive or negative potential pulses, a steadily applied voltage, a cyclic voltammogram, or other voltage combinations. In general, the voltage profile may be configured to reset or regenerate electrode surfaces to remove sensor drift caused by changes in electrode surface properties over time.

The application or adjustment of voltages based on a voltage profile may provide less sensor-to-sensor variability, allow sensors to operate for longer periods of time between sensor calibrations, and longer use of sensor apparatus because such voltage application or adjustment can reset or regenerate electrode surfaces back to their original surface characteristics. Regeneration of an ionic gradient in electrodes using the apparatus, systems, and methods described herein can reduce or eliminate sensor drift of the electrodes over time and that the electrodes remain reliable. Accordingly, apparatus, systems, and methods that can adjust at least one electrode to regenerate one or more electrodes as described herein may provide more accurate and reliable analyte sensing.

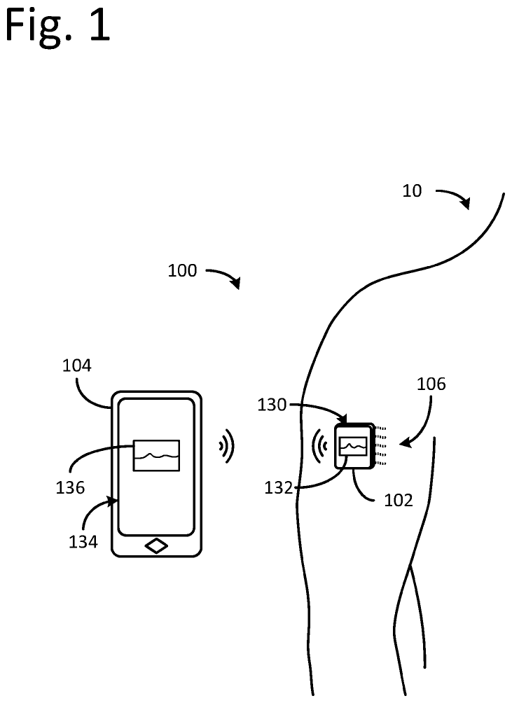

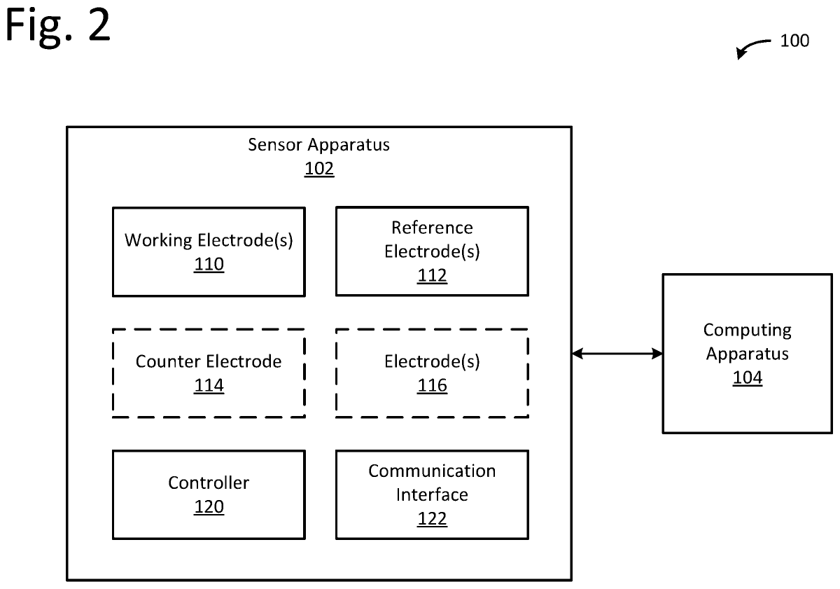

An analyte sensing system is depicted in FIGS. 1 and 2. FIG. 1 shows schematic diagram of an analyte sensing system 100 including an analyte sensor apparatus 102 and a computing apparatus 104 during use. FIG. 2 shows a schematic block diagram of the analyte sensing system 100.

The analyte sensing system 100 includes the analyte sensor apparatus 102 for detecting analyte in a target environment 10. As shown, the target environment 10 is a person or patient. The target environment 10 may be on or in the person or patient. However, the target environment 10 may include any environment where detection of analyte levels or concentrations may be desired. For example, the analyte sensor apparatus 102 may be used to detect analytes that may include reagents in chemical processes, pollutants (e.g., atmospheric or aquatic), health markers (e.g., cholesterol, triglycerides, iron, vitamins, etc.), any substance of a comprehensive metabolic panel (e.g., substances in blood including glucose, calcium, sodium, potassium, carbon dioxide, chloride, albumin, total protein, alkaline phosphatase, alanine transaminase, aspartate aminotransferase, bilirubin, blood urea nitrogen, and creatinine), etc. Accordingly, the target environment 10 may include, for example, a manufacturing facility, a body of water, a particular geographical area, a patient, human samples (e.g., blood, urine, saliva, etc.), environmental samples (e.g., air, water, soil, vegetation), cell cultures, food samples, etc. Furthermore, the analyte sensor apparatus 102 may be an implantable medical device, a remote sensor, a wearable device, hospital equipment, lab equipment, etc.

The analyte sensor apparatus 102 includes a plurality of electrodes 106. Each of the plurality of electrodes 106 may be configured to provide signals based on the target environment 10 that can be used to determine an analyte level or analyte concentration in the target environment 10. Each of the plurality of electrodes 106 may provide an electrical interface between the analyte sensor apparatus and the target environment. Signals provided by the plurality of electrodes 106 may include baseline signals, analyte signals, error condition signals, or other signals that can be used to determine an analyte level in the target environment.

The plurality of electrodes 106 may include any suitable type of electrode to provide an electrical interface, act as an electrochemical biotransducer, and/or be part of an electronic biotransducer. Electrodes may include a biorecognition element that selectively reacts with a target analyte and produces an electrical signal that is proportional to the analyte concentration. Electrodes may be used in amperometric, potentiometric, or other electrochemical sensor apparatus.

Amperometric sensor apparatus may be used to detect a change in current as a result of electrochemical oxidation or reduction. Typically, a bioreceptor molecule is immobilized on one or more working electrodes (e.g., gold, carbon, platinum, etc.). A potential applied between the one or more working electrodes and a reference electrode (e.g., silver/silver-chloride) may be fixed at a given value while the current is measured with respect to time. The applied potential may be the driving force for an electron transfer reaction. The current produced as a result of the applied potential may be a direct measure of the rate of electron transfer. Accordingly, the measured current may reflect the reaction occurring between the bioreceptor molecule and the analyte where the current is limited by the mass transport rate of the analyte to the electrode.

Potentiometric sensor apparatus may be used to measure a potential or charge accumulation. Biotransducers used for potentiometric sensing may include one or more working electrodes (e.g., an ion selective electrode) and a reference electrode. Each of the one or more working electrodes may include a membrane or surface that selectively interacts with a charged ion of interest (e.g., the analyte), causing the accumulation of a charge potential on each of the one or more working electrodes when compared to the reference electrode. The reference electrode may provide a constant half-cell potential that is unaffected by analyte concentration. A voltmeter may be used to measure the potential between the each of the one or more working electrodes and the reference electrode when no significant current flows between them. The potentiometric response may be governed by the Nernst equation such that the measured potential is proportional to a logarithm of the concentration of the analyte.

Electronic biotransducers may be based on field-effect transistors (FETs). A FET is a type of transistor that uses an electric field to control the conductivity of a channel (e.g., a region depleted of charge carriers) between two electrodes (e.g., the source and drain) in a semiconducting material. Control of the conductivity of the channel may be achieved by varying the electric field potential, relative to the source and drain electrode, at a third electrode, known as the gate. Depending on the configuration and doping of the semiconducting material, the presence of a sufficient positive or negative potential at the gate electrode can either attract charge carriers (e.g., electrons) or repel charge carriers in the conduction channel resulting in a change in drain current. Accordingly, as a target analyte accumulates on the gate electrode, a change in drain current can be determined and compared to a reference electrode to determine an analyte level or concentration.

Examples of various types of electrodes discussed herein are exemplary and do not provide an exhaustive list of electrodes that may be used in the apparatus, systems, and methods described herein. In general, the apparatus, systems, and methods described herein include at least one working electrode (e.g., an electrode sensitive to a target analyte) and at least one reference electrode (e.g., an electrode that is not sensitive to the target analyte). Signals from working electrodes may be compared to one or more refence baseline signals provided by one or more reference electrodes to determine the portion of the signals from the working electrodes that corresponds to the analyte level or concentration of the target environment (e.g., target environment 10). However, electrodes may be subject to noise, drift, or other error conditions that can impact the accuracy of sensor output results (e.g., analyte levels or concentration). To correct for drift or other measurement errors, apparatus, systems, and methods described herein may adjust a voltage of at least one electrode of the plurality of electrodes 106 to regenerate one or more electrodes of the plurality of electrodes 106.

The plurality of electrodes 106 include one or more working electrodes 110 and one or more reference electrodes 112. The one or more working electrodes 110 may each provide an analyte signal based on a presence of the analyte in the target environment 10. The one or more working electrodes 110 may be configured to interact with the analyte in such a way that the provided analyte signals vary in proportion to the concentration of the analyte in the target environment 10. For example, the one or more working electrodes 110 may include a bioreceptor that causes molecules of the analyte to accumulate on the one or more working electrodes 110 in proportion to the concentration of the analyte in the target environment 10. Accordingly, the analyte signals may vary in proportion to the concentration of the analyte in the target environment 10.

The reference electrodes 112 may each provide a baseline signal of the target environment 10. In general, the reference electrodes 112 do not react to the analyte. In other words, the baseline signals provided by the reference electrodes 112 are unaffected by the presence of the analyte in the target environment 10. Instead, the baseline signals may be representative of noise and other conditions of the target environment 10 that may impact signals provided by any of the plurality of electrodes 106. The baseline signals may be used in conjunction with the analyte signals to determine the analyte level or concentration of the target environment. Additionally, the baseline signals may be used to remove noise and other artifacts from the analyte signals.

The sensor apparatus 102 may optionally include other electrodes in addition to any of the working electrodes 110 or the reference electrodes 112. For example, the sensor apparatus 102 may optionally include a counter electrode 114. Generally, a counter electrode may refer to an element used as a current path to complete a circuit of an analyte sensor apparatus that uses current flow between electrodes to sense an analyte. For example, the counter electrode 114 may be used for cyclic voltammetry, linear sweep voltammetry, and other electrochemical techniques. The counter electrode 114 may be operatively coupled to a power supply to provide a voltage or current within the target environment 10 or to other components of the analyte sensor apparatus 102. Accordingly, power supplied to the plurality of electrodes 106 may be reduced. Similarly, a reduction in power requirements may allow a reduction in the size of the plurality of electrodes 106. Such voltage or current may be supplied primarily by the counter electrode 114 instead of the working electrodes 110 or the reference electrodes 112.

Furthermore, the sensor apparatus may optionally include one or more other additional electrodes 116. The one or more electrodes 116 may include any suitable electrode to provide noise, drift, or other error correction. The electrode 116 may include a blank electrode. A blank electrode may be an electrode that responds to background noise of the target environment 10 but does not otherwise react (chemically or otherwise) to the target environment. Error correction signals provided by blank electrodes may be used to correct for background noise that may affect reference electrodes 112.

Each of the plurality of electrodes 106 may be subject to drift. Drift or sensor drift may refer to an increase in measurement error of electrodes (e.g., the plurality of electrodes 106) in the target environment (e.g., target environment 10) over time. In other words, the magnitude of the measurement error of an electrode typically increases the longer the electrode remains in the target environment without calibration or other adjustments. To correct drift of an electrode without removal of the electrode the from the target environment 10, a voltage of at least one of the plurality of electrodes may be adjusted based on a voltage profile. Such voltage may be adjusted by a controller or computing apparatus.

The analyte sensor apparatus 102 may also include a controller 120 operatively coupled to the plurality of electrodes 106. The controller may be configured to a plurality of electrode signals from the plurality of electrodes 106. The plurality of electrode signals may include one or more analyte signals, baseline signals, or other signals provided by the plurality of electrodes 106. The controller 120 may receive signals from the plurality of electrodes 106 via any suitable wired or wireless connection.

The controller 120 may be configured to adjust a voltage of at least one electrode of the plurality of electrodes 106 based on a voltage profile to regenerate one or more electrodes of the plurality of electrodes 106 while the at least one electrode is disposed in the target environment 10. The at least one electrode may be any electrode of the plurality of electrodes 106 (e.g., one of the working electrodes 110, reference electrodes 112, the counter electrode 114, or other electrode 116).

The voltage profile may indicate or guide the adjustment of the voltage of the at least one electrode. In one or more embodiments, the voltage profile may include a series of voltage pulses. An adjustment of the voltage based on a voltage profile that includes a series of voltage pulses may cause the voltage of the at least one electrode to be suddenly increased or decreased for short periods of time for a predetermined number of times. In one or more embodiments, the voltage profile includes a series of cyclic voltammograms. A voltammogram may refer to steadily increasing or decreasing the voltage of an electrode from an initial voltage over a given time period followed by steadily bringing the voltage back to the initial voltage. An adjustment of the voltage based on a voltage profile that includes a series of voltammograms may cause the at least one electrode to be subject to a predetermined number of sequential voltammograms.

Adjustment of the voltage of the at least one electrode may regenerate any one or more of the plurality of electrodes 106. In other words, adjustment of the voltage of the at least one electrode may regenerate the at least one electrode, the one or more working electrodes 110, the one or more reference electrodes 112, the counter electrode 114, or the other one or more electrodes 116. Additionally, the at least one electrode may include any one or more of the plurality of electrodes 106. In one or more embodiments, the at least one electrode may include a regeneration electrode (e.g., the other electrode 116). Furthermore, the controller 120 may be configured to adjust a voltage between the regeneration electrode and one or more electrodes of the plurality of electrodes 106.

In one or more embodiments, the at least one electrode is a reference electrode configured to provide a baseline signal (e.g., reference electrode 112). In one or more embodiments, the at least one electrode is a working electrode configured to provide an analyte signal (e.g., working electrode 110). In one or more embodiments, the at least one electrode is configured to provide an adjustment signal (e.g., an adjustment of the voltage based on a voltage profile as described herein). The at least one electrode may include one or more electrode types such as, for example, a silver/silver chloride electrode, a silver chloride electrode, a silver/silver sulfide electrode, a silver/silver sulfate electrode, a silver/silver salt electrode, an iridium oxide electrode, a magnesium-based electrode, a solid-state configured reference electrode, solid-state ionic liquid reference electrode, etc. In one or more embodiments, the at least one electrode includes a silver/silver chloride electrode.

The controller 120 may be configured to adjust the voltage of the at least one electrode in response to a regeneration criterion being met. The regeneration criterion may include, for example, a threshold period of time elapsing, an estimated drift exceeding a threshold, an electrode signal differing from another electrode signal in excess of a difference threshold, etc. The period of time may be measured from a time when the plurality of electrodes 106 were placed in the target environment, from a time of the most recent calibration, or from a time of the most recent electrode regeneration. In one or more embodiments, the controller 120 may be configured to determine that the regeneration criterion has been met.

The controller 120 may further be configured to provide one or more analyte levels based on the plurality of electrode signals. For example, the controller 120 may be configured to provide the one or more analyte levels based on one or more baseline signals and one or more analyte signals. Further, for example, the one or more analyte levels may be provided or determined based on a difference between the baseline signal and each of the one or more analyte signals.

Still further, the controller 120 may be configured to provide an error correction factor based on the baseline signal and at least one error correction signal. The error correction factor may be provided or determined based on a difference between the baseline signal and the at least one error correction signal. The error correction signal may be provided by one of the plurality of electrodes 106 (e.g., other electrode 116) or may be determined based on any one or more of the plurality of electrode signals. Additionally, the error correction factor may be provided or determined based on any subtraction or differential method such as digital signal processing, noise subtraction, blind spot suppression, etc.

In some embodiments, the controller 120 may be an analog controller that includes one or more resistors, capacitors, operational amplifiers, comparators, filters, differential amplifiers, or other analog circuitry to provide various outputs without the use of digital circuitry, digital logic, or a processor. Such analog controller may be configured to provide one or more analyte levels based on the baseline signal and the one or more analyte signals. For example, a differential amplifier may provide a signal representative of the analyte level by providing the difference between an analyte signal and the baseline signal. Additional analogue circuitry may be used to provide the error correction factor and output the adjusted analyte levels.

In some embodiments, the controller 120 may additionally or alternatively include one or more processors, logic gates, or other digital circuitry to determine analyte levels and reference baselines. The controller 120 may include data storage for data storage and access to processing programs or routines that may be employed to carry out the techniques, processes, and algorithms for detecting an analyte in a target environment and/or correcting drift. For example, processing programs or routines may include programs or routines for adjusting a voltage of at least one electrode, regenerating one or more electrodes, determining one or more analyte levels, determining baselines, determining reference baselines, determining adjusted analyte levels, filtering background noise, computational mathematics, matrix mathematics, Fourier transforms, compression algorithms, calibration algorithms, inversion algorithms, signal processing algorithms, normalizing algorithms, deconvolution algorithms, averaging algorithms, standardization algorithms, comparison algorithms, vector mathematics, or any other processing required to implement one or more embodiments as described herein. Further, for example, processing programs or routines may not require direct active management. The exact configuration of the processing program(s) is not limiting and essentially any processing program capable of providing suitable processing capabilities, including any artificial intelligence, machine learning algorithms, and/or smart learning algorithms, may be used.

The analyte sensor apparatus 102 may also include a communication interface 122 to communicate with one or more external devices. The communication interface 122 may include any suitable hardware or devices to provide wired or wireless communication with the one or more external devices. For example, the communication interface 122 may include one or more of a receiver, transmitter, transceiver, ethernet port, Universal Serial Bus (USB) port, cables, controller, or other device to facilitate wired or wireless communication. The communication interface 122 may facilitate communication using any suitable protocol or protocols. For example, the communication interface 122 may utilize Ethernet, Recommended Standard 232, Universal Asynchronous Receiver Transmitter or Universal Synchronous Asynchronous Receiver Transmitter (UART/USART), USB, BLUETOOTH, Wi-Fi, Near Field Communication (NCF), tissue conduction, etc. The communication interface 122 may allow communication between the analyte sensor apparatus 102 and a computing apparatus such as the computing apparatus 104.

The system 100 may also include the computing apparatus 104. The computing apparatus 104 may be, for example, any fixed or mobile computer system (e.g., a personal computer, a tablet computer, a mobile device, a cellular phone, a wearable device, a cloud computing source that may not require direct active management, an integrated circuit, etc.). The exact configuration of the computing apparatus 104 is not limiting and essentially any device capable of providing suitable computing capabilities and control capabilities, including any artificial intelligence, machine learning algorithms, and/or smart learning algorithms, may be used (e.g., to control analyte sensing of the analyte sensor apparatus 102, to acquire data such as sensor data, to determine various parameters such as analyte levels, error correction factors, adjusted analyte levels, etc., to adjust analyte sensing of the analyte sensor apparatus 102, to report the acquired data and/or determined parameter(s), etc.). Further, various peripheral devices, such as a computer display, mouse, keyboard, memory, printer, scanner, etc. are contemplated to be used in combination with the computing apparatus 104.

The computing apparatus 104 may be operatively coupled to the analyte sensor apparatus 102. For example, the computing apparatus 104 may be operatively coupled to the analyte sensor apparatus 102 via analog electrical connections, digital electrical connections, wireless connections, bus-based connections, network-based connections, internet-based connections, etc. The computing apparatus 104 can transmit data to and receive data from the analyte sensor apparatus 102. Data received from the analyte sensor apparatus 102 may include, for example, analyte signals, baseline signals, voltage profiles, reference baseline signals, analyte levels, error correction factors, adjusted analyte levels, electrode or biotransducer status information, sensor parameters, etc. The computing apparatus 104 may be configured execute processes and methods described herein using data received from the analyte sensor apparatus 102. For example, the computing apparatus may be configured to determine analyte levels, regeneration criterion, baselines, reference baselines, correction factors, adjusted analyte levels, etc. In one or more embodiments, the computing apparatus 104 may include artificial intelligence that is configured to determine analyte levels, regeneration criterion, baselines, reference baselines, correction factors, adjusted analyte levels, etc. Data transmitted by the computing apparatus 104 to the analyte sensor apparatus 102 may include commands, voltage profiles, regeneration criterion, sensor settings, thresholds, parameters, etc.

Additionally, the analyte sensor apparatus 102 and the computing apparatus 104 may each include display apparatus 130, 134, respectively, that may be configured to display data. For example, the display apparatus 130, 134 may be configured to display one or more of the analyte levels, regeneration progress, regeneration criterion, reference baselines, error correction factors, adjusted analyte levels, biotransducer status information, sensor parameters, etc. The display apparatus 130, 134 may include any apparatus capable of displaying information to a user, such as a graphical user interface 132, 136 including one or more metrics indicative of medical system therapy (e.g., cardiac conduction system therapy, neurostimulation, etc.) benefit, one or more metrics indicative of analyte signals, baseline signals, reference baseline signals, error correction signals, analyte levels, error correction factors, adjusted analyte levels, biotransducer status information, sensor parameters, textual instructions, graphical depictions the target environment, graphical depictions or actual images of the plurality of electrodes 106, etc. Further, the display apparatus 130, 134 may include a liquid crystal display, an organic light-emitting diode screen, a touchscreen, a cathode ray tube display, etc.

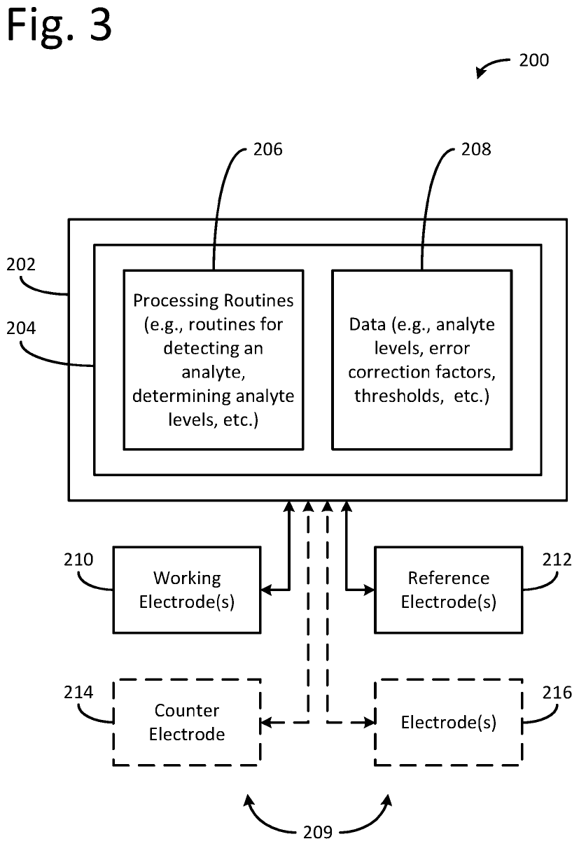

A schematic block diagram of another analyte sensing system 200 according to embodiments described herein is shown in FIG. 3. The analyte sensing system 200 may include a computing apparatus or processor 202 and a plurality of electrodes 209. Generally, the plurality of electrodes 209 may be operatively coupled to the computing apparatus 202 and may include any suitable circuits or devices configured to provide signals for detecting an analyte in a target environment (e.g., baseline signals, analyte signals, and error condition signals) similar to the plurality of electrodes 106 of FIGS. 1 and 2.

The plurality of electrodes 209 may include one or more working electrodes 210 and one or more reference electrodes 212. The one or more working electrodes 210 may be configured to provide an analyte signal based on the presence of the analyte in the target environment and may include any of the features described herein with regard to working electrode 110. The one or more reference electrodes 212 may be configured to provide baseline signals of the target environment and may include any of the features described herein with regard to reference electrodes 112.

The analyte sensing system 200 may optionally include a counter electrode 214. The first reference electrode 212 may be operatively coupled to the counter electrode 214. The counter electrode 214 may be configured to provide power to the plurality of electrodes 209 and may include any of the features described herein with regard to counter electrode 114. The analyte sensing system 200 may optionally include one or more electrodes 216. The one or more electrodes 216 may include any of the features described herein with regard to the electrode 116. For example, the one or more electrodes 216 may include a regeneration electrode, a blank electrode, etc.

Further, the computing apparatus 202 includes data storage 204. Data storage 204 allows for access to processing programs or routines 206 and one or more other types of data 208 that may be employed to carry out the techniques, processes, and algorithms for charging a battery or one or more electrochemical cells. For example, processing programs or routines 206 may include programs or routines for adjusting/applying voltages, providing/determining voltage profiles, resetting/regenerating electrodes, providing/determining analyte levels, providing/determining baselines, providing/determining reference baselines, providing/determining error correction factors, outputting/determining adjusted analyte levels, filtering background noise, transmitting data, receiving data, determining thresholds, computational mathematics, matrix mathematics, Fourier transforms, compression algorithms, calibration algorithms, image construction algorithms, inversion algorithms, signal processing algorithms, normalizing algorithms, deconvolution algorithms, averaging algorithms, standardization algorithms, comparison algorithms, vector mathematics, or any other processing required to implement one or more embodiments as described herein.

Data 208 may include, for example, voltage profile data, electrode data, signal data, analyte level data, baseline data, reference baseline data, error correction factor data, biotransducer settings/parameters, calibration data, resistance calculations, device settings, error bit states, historical data, thresholds, arrays, meshes, grids, variables, counters, statistical estimations of accuracy of results, results from one or more processing programs or routines employed according to the disclosure herein (e.g., detecting an analyte in a target environment, determining adjusted analyte levels, etc.), or any other data that may be necessary for carrying out the one or more processes or techniques described herein.

In one or more embodiments, the analyte sensing system 200 may be controlled using one or more computer programs executed on programmable computers, such as computers that include, for example, processing capabilities (e.g., microcontrollers, programmable logic devices, artificial intelligence, smart learning algorithms, integrated circuits, cloud computing, etc.), data storage (e.g., volatile or non-volatile memory and/or storage elements), input devices, and output devices. Program code and/or logic described herein may be applied to input data to perform functionality described herein and generate desired output information. The output information may be applied as input to one or more other devices and/or processes as described herein or as would be applied in a known fashion.

The programs used to implement the processes described herein may be provided using any programmable language, e.g., a high-level procedural and/or object orientated programming language that is suitable for communicating with a computer system or machine learning algorithm. Any such programs may, for example, be stored on any suitable device, e.g., a storage media, readable by a general or special purpose program, computer or a processor apparatus for configuring and operating the computer when the suitable device is read for performing the procedures described herein. In other words, at least in one embodiment, the analyte sensing system 200 may be controlled using a computer readable storage medium, configured with a computer program, where the storage medium so configured causes the computer to operate in a specific and predefined manner to perform functions described herein.

The computing apparatus 202 may be, for example, any fixed or mobile computer system (e.g., a personal computer, minicomputer, a smart device, etc.). The exact configuration of the computing apparatus is not limiting and essentially any device capable of providing suitable computing capabilities and control capabilities (e.g., control electrode output such as voltage, current, photon, or other sensing outputs; the acquisition of data, such as sensor data; etc.) may be used. Additionally, the computing apparatus 202 may be incorporated in a housing of the analyte sensing system 200. Further, various peripheral devices, such as a computer display, mouse, keyboard, memory, printer, scanner, etc. are contemplated to be used in combination with the computing apparatus 202. Further, in one or more embodiments, the data 208 (e.g., voltage profile data, signal data, analyte level data, baseline data, reference baseline data, error correction factor data, biotransducer settings/parameters, calibration data, etc.) may be analyzed by a user, used by another machine that provides output based thereon, etc. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by computing apparatus 202 described herein. Also, as described herein, a file in user-readable format may be any representation of data (e.g., ASCII text, binary numbers, hexadecimal numbers, decimal numbers, audio, graphical) presentable on any medium (e.g., paper, a display, sound waves, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware that is to be used to implement the processes described herein shall not be limiting on the scope of the systems, processes or programs (e.g., the functionality provided by such systems, processes or programs) described herein.

The techniques described in this disclosure, including those attributed to the systems, or various constituent components, may be implemented, at least in part, in hardware, software, firmware, or any combination thereof. For example, various aspects of the techniques may be implemented by the computing apparatus 202, which may use one or more processors such as, e.g., one or more microprocessors, DSPs, ASICs, FPGAS, CPLDs, microcontrollers, or any other equivalent integrated or discrete logic circuitry, as well as any combinations of such components, image processing devices, or other devices. The term “processing apparatus,” “processor,” or “processing circuitry” may generally refer to any of the foregoing logic circuitry, alone or in combination with other logic circuitry, or any other equivalent circuitry. Additionally, the use of the word “processor” may not be limited to the use of a single processor but is intended to connote that at least one processor may be used to perform the techniques and processes described herein.

Such hardware, software, and/or firmware may be implemented within the same device or within separate devices to support the various operations and functions described in this disclosure. In addition, any of the described components may be implemented together or separately as discrete but interoperable logic devices. Depiction of different features, e.g., using block diagrams, etc., is intended to highlight different functional aspects and does not necessarily imply that such features must be realized by separate hardware or software components. Rather, functionality may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by the computing apparatus 202 to support one or more aspects of the functionality described in this disclosure.

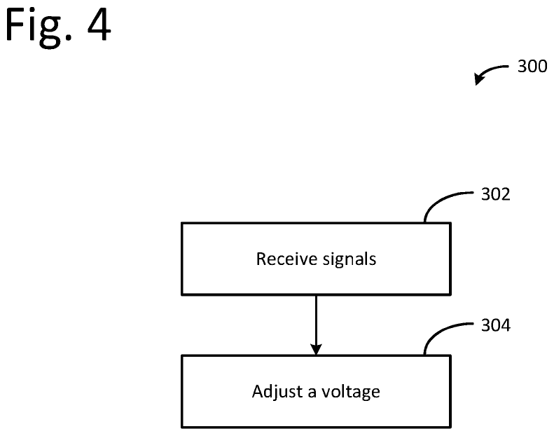

A method or process 300 of detecting an analyte in a target environment is shown in FIG. 4. Although described in regard to analyte sensor apparatus 102 of FIGS. 1 and 2 or analyte sensing system 200 of FIG. 3, the method 300 may be carried out using any suitable analyte sensing apparatus or system.

The method 300 may include receiving a plurality of electrode signals 302. The plurality of electrode signals may include one or more analyte signals of the target environment (e.g., target environment 10) from one or more working electrodes (e.g., working electrodes 110 or 210) disposed in the target environment and one or more baseline signals of the target environment from one or more reference electrodes (e.g., reference electrodes 112 or 212). In one or more embodiments, the plurality of electrode signals further includes signals from one or more additional electrodes (e.g., electrode 116 or 216). For example, a blank baseline signal provided by a blank electrode.

The method 300 may further include adjusting a voltage of at least one electrode of the plurality of electrodes based on a voltage profile to regenerate one or more electrodes of the plurality of electrodes while the at least one electrode is disposed in the target environment 304. In other words, the voltage of the at least one electrode may be adjusted and the one or more electrodes may be regenerated without removing the electrodes from the target environment. The voltage profile may include a single voltage pulse, a series of voltage pulses, a voltammogram, a series of cyclic voltammograms, a regeneration voltage, or other voltage levels or changes configured to regenerate the one or more electrodes. Adjustment of the voltage of the at least one electrode may continue until a regeneration threshold has been met. The regeneration threshold may be based on, for example, a target electrode current, a target electrode charge, or some other parameter that indicates the one or more electrodes have been regenerated.

The method 300 may further include adjusting the voltage of the at least one electrode in response to a regeneration criterion being met. In other words, the adjustment of the at least one electrode based on the voltage profile may occur or be initiated in response to the regeneration criterion being met. The regeneration criterion may include a period of time elapsing from an initialization event (e.g., a most recent calibration or regeneration of one or more electrodes), an estimated drift exceeding a threshold, a scheduled regeneration time, a noise level exceeding a threshold, a signal difference between a reference electrode (e.g., reference electrode 112) and another electrode (e.g., electrode 116) exceeding a threshold, etc. The method 300 may further include determining the regeneration criterion has been met. The determination that the regeneration criterion has been met may be based on the plurality of electrode signals, the elapsed period of time from the initialization event, the scheduled regeneration time, etc.

The method 300 may further include displaying an alert in response to the regeneration criterion being met. The alert may indicate that electrode regeneration (e.g., adjusting the voltage of the at least one electrode) is in progress or is about to begin. The alert may be displayed on at least one of the display apparatus (e.g., display apparatus 130 or 134) and/or graphical user interfaces (e.g., graphical user interface 132 or 136).

The method 300 may further include providing an analyte level based on the plurality of electrode signals. The analyte level may be provided by a sensing apparatus (e.g., sensing apparatus 102) or a computing apparatus (e.g., computing apparatus 104 or 202). In one or more embodiments, the analyte level may be provided by the sensing apparatus as a signal representative of the analyte level (e.g., an analyte level signal). The analyte level signal may be provided using a communication interface (e.g., communication interface 122). The analyte level signal may be a wired or wireless signal. In one or more embodiments, the analyte signal may be provided by a display (e.g., displays 130, 134). Additionally, providing the analyte level may include displaying one or more adjusted analyte levels. Displaying the one or more analyte levels or adjusted analyte levels may include displaying such analyte levels on at least one of the display apparatus (e.g., display apparatus 130 or 134) and/or graphical user interfaces (e.g., graphical user interface 132 or 136). The one or more analyte levels or adjusted analyte levels may be displayed as a number, graph, or other visual representation of the adjusted analyte levels.

To provide the one or more analyte levels, the method 300 may further include determining a reference baseline or providing a reference baseline signal. A reference baseline may be determined based on a single baseline signal, a plurality of baseline signals, a blank electrode signal, or other reference or error correction signals. The reference baseline may be an average baseline. In other words, the reference baseline may be an average of baseline signals provided by reference electrodes (e.g., the reference electrodes 112) of the sensor apparatus. Determining a reference baseline or providing a reference baseline signal may also include one or more additional techniques to allow noise and other errors to be removed from the one or more analyte signals to provide or determine a more accurate analyte level. The differences between the one or more analyte signals and an actual analyte level in the target environment may be caused by electrical noise, magnetic noise, light induced artifacts, movement induced artifacts, non-faradaic currents, faradaic non-analyte induced currents, background environment conductivity, electrochemical drift, etc. Artificial intelligence may recognize the cause of the difference between the one or more analyte signals and the actual analyte level in the target environment, and may further correct for the difference. In some embodiments, providing the analyte level may include providing the result of any suitable subtraction or differentiation method applied to the one or more analyte signals and the reference baseline signals such as, for example, digital signal processing, noise subtraction, blind spot suppression, etc. In some embodiments, the specifics of the differentiation method and the specific parameters and factors used to determine when to implement the method will be controlled by artificial intelligence, a machine learning algorithm, a smart learning algorithm, etc. The artificial intelligence or learning algorithm may be trained to minimize signal drift, maximize sensor stability, etc.

In at least one embodiment, the method may further include using at least one of an artificial intelligence platform and a machine learning algorithm to perform at least one of: adjusting the voltage of the at least one electrode of the plurality of electrodes, determining the regeneration criterion, and adjusting the voltage of the at least one electrode in response to the regeneration criterion being met.

The invention is defined in the claims. However, below there is provided a non-exhaustive list of non-limiting examples. Any one or more of the features of these examples may be combined with any one or more features of another example, embodiment, or aspect described herein.

Example Ex1: An analyte sensor apparatus for detecting an analyte in a target environment comprising: a plurality of electrodes to provide a plurality of electrode signals based on the target environment while disposed in the target environment; and a controller operatively coupled to the plurality of electrodes and configured to: receive the plurality of electrode signals; adjust a voltage of at least one electrode of the plurality of electrodes based on a voltage profile to regenerate one or more electrodes of the plurality of electrodes while the at least one electrode is disposed in the target environment.

Example Ex2: The apparatus as in example Ex1, wherein controller is configured to adjust the voltage of the at least one electrode in response to a regeneration criterion being met.

Example Ex3: The apparatus as in example Ex1, wherein the controller comprises one or more processors and is further configured to: determine a regeneration criterion has been met; and adjust the voltage of the at least one electrode in response to the regeneration criterion being met.

Example Ex4: The apparatus as in any one of examples Ex1 to Ex3, wherein the voltage profile comprises a series of voltage pulses.

Example Ex5: The apparatus as in any one of examples Ex1 to Ex4, wherein the voltage profile comprises a series of cyclic voltammograms.

Example Ex6: The apparatus as in any one of examples Ex1 to Ex5, wherein the controller is further configured to select the voltage profile based on a type of the at least one electrode.

Example Ex7: The apparatus as in any one of examples Ex1 to Ex6, wherein the at least one electrode comprises a regeneration electrode and wherein the controller is configured to adjust a voltage between the regeneration electrode and the one or more electrodes.

Example Ex8: The apparatus as in any one of examples Ex1 to Ex7, wherein the at least one electrode comprises a reference electrode configured to provide a baseline signal.

Example Ex9: The apparatus as in any one of examples Ex1 to Ex8, wherein the at least one electrode comprises a working electrode configured to provide an analyte signal.

Example Ex10: The apparatus as in any one of examples Ex1 to Ex9, wherein the at least one electrode comprises a silver/silver chloride electrode.

Example Ex11: The apparatus as in any one of examples Ex1 to Ex10, wherein the analyte sensor apparatus comprises an implantable medical device.

Example Ex12: An analyte sensing system comprising: an analyte sensor apparatus for detecting an analyte in a target environment comprising a plurality of electrodes to provide a plurality of electrode signals based on the target environment while disposed in the target environment; and a computing apparatus comprising one or more processors and operatively coupled to the analyte sensor apparatus, the computing apparatus configured to: receive the plurality of electrode signals; and adjust a voltage of at least one electrode of the plurality of electrodes based on a voltage profile to regenerate one or more electrodes of the plurality of electrodes while the at least one electrode is disposed in the target environment.

Example Ex13: The system as in example Ex12, wherein the computing apparatus is configured to adjust the voltage of the at least one electrode in response to a regeneration criterion being met.

Example Ex14: The system as in any one of examples Ex12 or Ex13, wherein the voltage profile comprises a series of voltage pulses.

Example Ex15: The system as in any one of examples Ex12 or Ex14, wherein the voltage profile comprises a series of cyclic voltammograms.

Example Ex16: The system as in any one of examples Ex12 to Ex15, wherein the computing apparatus is further configured to select the voltage profile based on a type of the at least one electrode.

Example Ex17: The system as in any one of examples Ex12 to Ex16, wherein the at least one electrode comprises a regeneration electrode and wherein the computing apparatus is configured to adjust a voltage between the regeneration electrode and the one or more electrodes.

Example Ex18: The system as in any one of examples Ex12 to Ex17, wherein the at least one electrode comprises a reference electrode configured to provide a baseline signal.

Example Ex19: The system as in any one of examples Ex12 to Ex18, wherein the at least one electrode comprises a working electrode configured to provide an analyte signal.

Example Ex20: The system as in any one of examples Ex12 to Ex19, wherein the at least one electrode comprises a silver/silver chloride electrode.

Example Ex21: The system as in any one of examples Ex12 to Ex20, wherein the analyte sensor apparatus comprises an implantable medical device.

Example Ex22: The system as in any one of examples Ex12 to Ex21, wherein the analyte sensor apparatus comprises a wearable device.

Example Ex23: The system as in any one of examples Ex12 to Ex22, wherein the analyte sensor apparatus further comprises a communication interface configured to wirelessly transmit the plurality of electrode signals to the computing apparatus.

Example Ex24: The system as in any one of examples Ex12 to Ex23, wherein the computing apparatus further comprises a display and wherein the computing apparatus is further configured to display an electrode regeneration alert in response to a regeneration criterion being met.

Example Ex25: A method to reduce sensor drift of an analyte sensor apparatus disposed in a target environment, the method comprising: receiving a plurality of electrode signals from a plurality of electrodes; and adjusting a voltage of at least one electrode of the plurality of electrodes based on a voltage profile to regenerate one or more electrodes of the plurality of electrodes while the at least one electrode is disposed in the target environment.

Example Ex26: The method as in example Ex25, wherein the voltage of the at least one electrode is adjusted in response to a regeneration criterion being met.

Example Ex27: The method as in example Ex25, further comprising: determining a regeneration criterion of the at least one electrode has been met; and adjusting the voltage of the at least one electrode in response to the regeneration criterion being met.

Example Ex28: The method as in any one of examples Ex25 to Ex27, wherein the voltage profile comprises a series of voltage pulses.

Example Ex29: The method as in any one of examples Ex25 to Ex28, wherein the voltage profile comprises a series of cyclic voltammograms.

Example Ex30: The method as in any one of examples Ex25 to Ex29, further comprising selecting the voltage profile based on a type of the at least one electrode.

Example Ex31: The method as in any one of examples Ex25 to Ex30, wherein the at least one electrode comprises a regeneration electrode and wherein adjusting the voltage of the at least one electrode comprises adjusting a voltage between the regeneration electrode and the one or more electrodes.

Example Ex32: The method as in any one of examples Ex25 to Ex31, wherein the at least one electrode comprises a reference electrode configured to provide a baseline signal.

Example Ex33: The method as in any one of examples Ex25 to Ex32, wherein the at least one electrode comprises a working electrode configured to provide an analyte signal.

Example Ex34: The method as in any one of examples Ex25 to Ex33, wherein the at least one electrode comprises a silver/silver chloride electrode.

Example Ex35: The method as in any one of examples Ex25 to Ex33, wherein the reference electrode comprises a solid-state ionic liquid electrode. The solid-state ionic liquid reference electrode is configured to provide at least one of a baseline, analyte, and adjustment signal. The solid-state ionic liquid electrode may resemble those described in US20180024087A1 or U.S. Pat. No. 9,874,539B2.

Example Ex36: The method as in any one of examples Ex25 to Ex35, further comprising displaying an electrode regeneration alert in response to a regeneration criterion being met.

Example Ex37: The apparatus, system, or method as in any one of examples Ex25 to Ex36, wherein the voltage of the at least one electrode is adjusted based on the voltage profile until a regeneration threshold has been met.

Example Ex38: The method as in any one of examples Ex25 to Ex37, wherein the method further comprises using at least one of an artificial intelligence platform and a machine learning algorithm to perform at least one of: adjusting the voltage of the at least one electrode of the plurality of electrodes, determining the regeneration criterion, and adjusting the voltage of the at least one electrode in response to the regeneration criterion being met.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all of the listed elements or a combination of any two or more of the listed elements.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

1. An analyte sensor apparatus for detecting an analyte in a target environment comprising:
a plurality of electrodes to provide a plurality of electrode signals based on the target environment while disposed in the target environment; and
a controller operatively coupled to the plurality of electrodes and configured to:
receive the plurality of electrode signals; and
adjust a voltage of at least one electrode of the plurality of electrodes based on a voltage profile to regenerate one or more electrodes of the plurality of electrodes while the at least one electrode is disposed in the target environment.

2. The apparatus as in claim 1, wherein the controller is configured to adjust the voltage of the at least one electrode in response to a regeneration criterion being met.

3. The apparatus as in claim 1, wherein the voltage profile comprises one or more of a series of voltage pulses and a series of cyclic voltammograms.

4. The apparatus as in claim 1, wherein the controller is further configured to select the voltage profile based on a type of the at least one electrode.

5. The apparatus as in claim 1, wherein the at least one electrode comprises a regeneration electrode and wherein the controller is configured to adjust a voltage between the regeneration electrode and the one or more electrodes.

6. The apparatus as in claim 1, wherein the analyte sensor apparatus comprises an implantable medical device.

7. An analyte sensing system comprising:
an analyte sensor apparatus for detecting an analyte in a target environment comprising a plurality of electrodes to provide a plurality of electrode signals based on the target environment while disposed in the target environment; and
a computing apparatus comprising one or more processors and operatively coupled to the analyte sensor apparatus, the computing apparatus configured to:
receive the plurality of electrode signals; and
adjust a voltage of at least one electrode of the plurality of electrodes based on a voltage profile to regenerate one or more electrodes of the plurality of electrodes while the at least one electrode is disposed in the target environment.

8. The system as in claim 7, wherein the computing apparatus is configured to adjust the voltage of the at least one electrode in response to a regeneration criterion being met.

9. The system as in claim 7, wherein the voltage profile comprises one or more of a series of voltage pulses and a series of cyclic voltammograms.

10. The system as in claim 7, wherein the computing apparatus is further configured to select the voltage profile based on a type of the at least one electrode.

11. The system as in claim 7, wherein the at least one electrode comprises a regeneration electrode and wherein the computing apparatus is configured to adjust a voltage between the regeneration electrode and the one or more electrodes.

12. The system as in claim 7, wherein the analyte sensor apparatus comprises an implantable medical device.

13. The system as in claim 7, wherein the computing apparatus further comprises a display and wherein the computing apparatus is further configured to display an electrode regeneration alert in response to a regeneration criterion being met.

14. A method to reduce sensor drift of an analyte sensor apparatus disposed in a target environment, the method comprising:
receiving a plurality of electrode signals from a plurality of electrodes; and
adjusting a voltage of at least one electrode of the plurality of electrodes based on a voltage profile to regenerate one or more electrodes of the plurality of electrodes while the at least one electrode is disposed in the target environment.

15. The method as in claim 14, further comprising:
determining a regeneration criterion of the at least one electrode has been met; and
adjusting the voltage of the at least one electrode in response to the regeneration criterion being met.

16. The apparatus as in claim 2, wherein the regeneration criterion comprises one or more of: a threshold period of time elapsing, an estimated drift exceeding a threshold, and an electrode signal differing from another electrode signal in excess of a difference threshold.

17. The system as in claim 8, wherein the regeneration criterion comprises one or more of: a threshold period of time elapsing, an estimated drift exceeding a threshold, and an electrode signal differing from another electrode signal in excess of a difference threshold.

18. The method as in claim 15, wherein the regeneration criterion comprises one or more of: a threshold period of time elapsing, an estimated drift exceeding a threshold, and an electrode signal differing from another electrode signal in excess of a difference threshold.

19. The apparatus as in claim 1, wherein the at least one electrode comprises a reference electrode configured to provide a baseline signal and a blank electrode configured to provide an error correction signal, and wherein the controller is configured to provide an error correction factor based on a difference between the baseline signal and the error correction signal.

20. The system as in claim 7, wherein the at least one electrode comprises a reference electrode configured to provide a baseline signal and a blank electrode configured to provide an error correction signal, and wherein the computing apparatus is configured to provide an error correction factor based on a difference between the baseline signal and the error correction signal.
